(19) United States
(12) Patent Application Publication
(10) Pub. No.: US 2024/0335329 A1
(43) Pub. Date: Oct. 10, 2024

(54) NEGATIVE PRESSURE SUPER ABSORBENT SYSTEM WITH A PEEL OFF AND REPLACE FEATURE

(71) Applicant: KCI Manufacturing Unlimited Company, Westmeath (IE)

(72) Inventors: Jonathan G. REHBEIN, San Antonio, TX (US); Christopher J. CARROLL, San Antonio, TX (US)

(21) Appl. No.: 18/294,709

(22) PCT Filed: Jul. 15, 2022
(86) PCT No.: PCT/IB2022/056527

§ 371 Date: Feb. 2, 2024

Related U.S. Application Data
(60) Provisional application No. 63/228,312, filed on Aug. 2, 2021.

(51) Int. Cl. A61F 13/05; A61F 13/00; A61F 13/0206

(52) U.S. Cl. CPC A61F 13/05; A61F 13/0209; A61F 2013/00557

(57) ABSTRACT

A replaceable absorbent dressing system for a negative pressure wound therapy system includes a dressing and an absorbent pad. The dressing includes a perforated layer. The absorbent pad is detachably coupled to the dressing such that the absorbent pad can be separated from and reattached to the dressing while still forming a substantially airtight seal with the dressing. In at least one embodiment, the dressing further includes a foam material disposed on a skin facing side of the perforated layer.

### CROSS-REFERENCE TO RELATED APPLICATIONS

This application claims the benefit of priority to U.S. Provisional Application No. 63/228,312, filed on Aug. 2, 2021, which is incorporated herein by reference in its entirety.

### BACKGROUND

The present disclosure relates generally to wound dressings. More specifically, the present disclosure relates to wound dressings for negative pressure wound therapy systems.

NPWT is a therapeutic technique used to promote healing in acute or chronic tissue wounds. NPWT systems are configured to apply a negative pressure to a wound, through a dressing that is connected to the wound (e.g., to the skin surrounding the wound). The negative pressure may be utilized to remove infectious materials, and to help promote wound closure and healing. In order to maintain adequate pressure at the wound site, the dressing must form an airtight seal with the patient's tissue. The pump and fluid canister for the NPWT system must generally be sized to accommodate varying levels of wound exudate that is drawn away from the wound site. Additionally, in cases where absorbent materials are used in the dressing, the absorbent may become filled with fluids and wound exudate and need to be replaced. This process requires removal of the dressing from the patient, which may degrade the adhesive used to secure the dressing and renders the dressing unusable. It would be desirable to provide a device that allows for replacement of absorbent materials from the dressing without having to remove and replace the entire dressing system.

### SUMMARY

One implementation of the present disclosure is a replaceable absorbent dressing system for a negative pressure wound therapy system. The system includes a dressing and an absorbent pad. The dressing includes a perforated layer. The absorbent pad is detachably coupled to the dressing such that the absorbent pad can be separated from and reattached to the dressing while maintaining a substantially airtight seal with the dressing. In at least one embodiment, the dressing further includes a foam material disposed on a skin facing side of the perforated layer.

In some embodiments, the absorbent pad is detachably coupled to the dressing by intervening fastening layers including a first fastening layer and a second fastening layer that is detachably coupled to the first fastening layer. The first fastening layer may include a strip extending along a perimeter portion of the absorbent pad. The strip may include a plurality of stems that are configured to mechanically fasten the first fastening layer to the second fastening layer when the first fastening layer is pressed against the second fastening layer. In another embodiment, the first fastening layer may include a female interlocking member and the second fastening layer may include a male interlocking member configured to sealingly engage with the female interlocking member. In yet another embodiment, the first fastening layer includes a first fastening strip and a second fastening strip positioned on an opposite side of the absorbent pad as the first fastening strip. Both the first fastening strip and the second fastening strip may be oriented in a direction that is substantially parallel to a longest dimension of the absorbent pad.

In some embodiments, the absorbent pad forms part of an absorbent dressing. The absorbent dressing may further include an upper cover coupled to the absorbent pad and covering an upper surface of the absorbent pad, and a track pad coupled to the upper cover and fluidly coupled to an area of the upper cover that contains the absorbent pad. In one aspect, the track pad may further include a hydrophobic filter that substantially prevents fluid from passing through the track pad.

In some embodiments, the perforated layer includes a plurality of openings that fluidly couple the absorbent pad with a patient facing side of the dressing. In at least one embodiment, the absorbent pad is aligned with the perforated layer such that the absorbent pad is positioned above at least some of the plurality of openings.

In some embodiments, the absorbent pad is detachably coupled to the dressing by a gel adhesive.

Another implementation of the present disclosure is a negative pressure wound therapy system. The negative pressure wound therapy system includes a first dressing, a second dressing, a touch fastener, and a negative pressure source. The first dressing includes an absorbent material and a negative pressure interface. The second dressing includes a perforated layer. The touch fastener is disposed between the first dressing and the second dressing, the touch fastener configured to detachably couple the first dressing to the second dressing and form a substantially airtight seal between the first dressing and the second dressing. The negative pressure source is coupled to the negative pressure interface.

Yet another implementation of the present disclosure is a replaceable absorbent dressing. The absorbent dressing includes an upper cover, an absorbent material, and a touch fastener. The upper cover defines a recessed area on a patient facing side of the upper cover. The absorbent material is disposed within the recessed area. The touch fastener is coupled to the upper cover along a perimeter portion of the upper cover, and is configured to detachably couple the upper cover to a dressing and form a substantially airtight seal between the upper cover and the dressing.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE FIGURES

FIG. 1 is an exploded view of a dressing system for a negative pressure wound therapy system, according to an illustrative embodiment.

FIG. 2 is a perspective view of a lower dressing of the dressing system of FIG. 1.

FIG. 3 is a perspective view of the dressing system of FIG. 1.

FIG. 4 is a perspective view of a touch fastener for the dressing system of FIG. 1.

FIGS. 5A-5C are side views of a touch fastener in various stages of assembly, according to an illustrative embodiment.

FIG. 6A is a perspective view of a dressing system for a negative wound pressure therapy system under an applied pressure, according to an illustrative embodiment.

FIG. 6B is a perspective view of the dressing system of FIG. 6A after removing an absorbent pad from a lower dressing.

FIG. 6C is a perspective view of the dressing system of FIG. 6A during attachment of a second absorbent pad from a lower dressing.

FIG. 6D is a perspective view of the dressing system of FIG. 6A under an applied pressure after attaching a second absorbent pad to a lower dressing.

FIG. 7 is a flow diagram of a method of making a dressing system for a negative pressure wound therapy system, according to an illustrative embodiment.

### DETAILED DESCRIPTION

### Overview

Referring generally to the Figures, a dressing system is shown that includes multiple dressing portions, including a base dressing (e.g., base layer, etc.) that is configured to be placed in direct fluid communication with a wound site, and an absorbent dressing (e.g., an absorbent layer, a removable dressing, an absorbent pad, and absorbent pouch, etc.) in fluid communication with the base dressing. The absorbent dressing is removable from the base dressing and may be separated from the base dressing when the absorbent material in the absorbent dressing is saturated with fluid (e.g., wound exudate, etc.). In this way, the absorbent dressing functions as a removable canister for the dressing system that eliminates the need for a separate, pump mounted canister that is typically required in negative pressure wound therapy (NPWT) systems.

Additionally, the absorbent dressing may be sized to accommodate various amounts (e.g., rates, etc.) of fluid ingestion from the wound site, depending on the size and/or type of wound, healing progress, etc. Additionally, because the absorbent dressing is removable from the base dressing, the size of the absorbent dressing (e.g., the amount of absorbent material within the absorbent dressing) may be changed during treatment, without removing the base dressing from the patient's skin, or affecting the main integral seal between the skin and the dressing system.

In at least one embodiment, the base dressing includes a perforated layer and a foam material coupled to a patient facing side of the perforated layer. The perforated layer includes a plurality of openings (e.g., perforations, etc.) that fluidly couple the patient facing side of the perforated layer (e.g., the foam material) to the absorbent dressing. The absorbent material in the absorbent dressing is separated from the wound site by the foam material. In other words, the absorbent material is isolated from the wound site by the foam material, in an arrangement that reduces the skin's exposure to moisture and the associated risk of maceration during treatment.

In at least one embodiment, the dressing system can be used as part of an NPWT system. In such implementations, the absorbent dressing includes an upper cover housing the absorbent material, and a track pad coupled to a negative pressure interface along the upper cover. The track pad may include a hydrophobic filter that substantially prevents fluid from passing through the track pad such that all the wound exudate is captured by the absorbent material. Among other benefits, this arrangement for the dressing system eliminates the need for a separate canister, integrated with the pump, to protect the pump during operation, thereby simplifying the design of the pump system.

In some embodiments, the dressing system includes a touch fastener disposed between the base layer and the absorbent layer that is configured to detachably couple the absorbent layer to the base dressing and to form a substantially airtight seal between the absorbent dressing and the base dressing. Beneficially, the touch fastener allows a user or clinician to remove and replace the absorbent dressing without removing the base dressing from the patient's skin, and without sacrificing the integrity of the seal between the base dressing and the absorbent dressing. These and other features and advantages of the dressing system are described in detail below.

### Dressing Construction

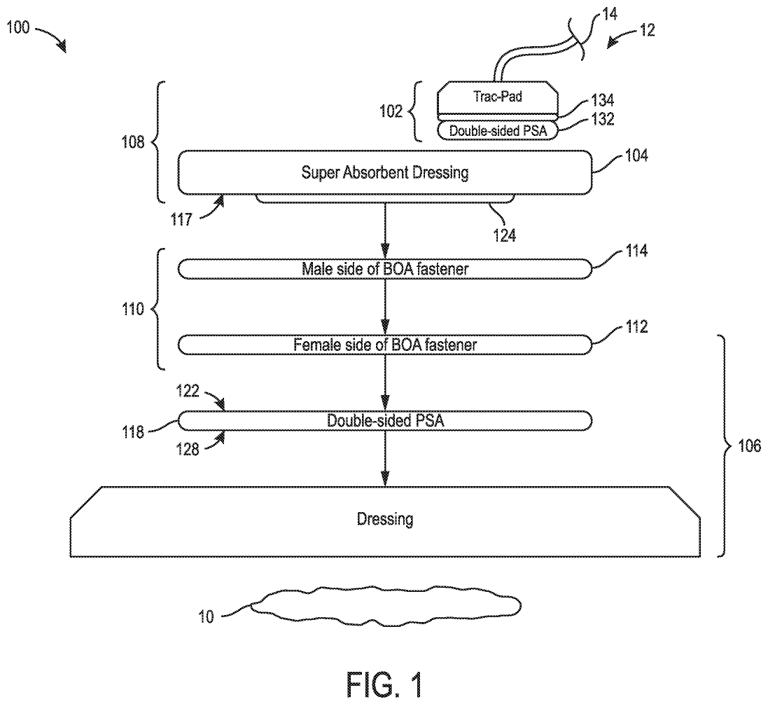

FIG. 1 shows a dressing system 100 with a removable and replaceable absorbent dressing, according to an illustrative embodiment. The dressing system 100 is applied to a wound site 10 (e.g., a patient area of treatment, etc.). The wound site 10 is shown to include a wound (e.g., incision, laceration, infected tissue, etc.) and periwound skin or other tissue surrounding the wound site 10. The dressing system 100 is configured to engage a negative pressure source with the wound site 10, and to form a substantially airtight and liquid tight seal with the wound site 10. The dressing system 100 is disposed centrally over the wound site 10 and covers the wound site 10. In other embodiments, the dressing system 100 may only cover a portion of the wound site 10 (e.g., a single leg of an incision, a first portion of an irregularly shaped wound, etc.).

According to an illustrative embodiment, the dressing system 100 is used with and/or is part of an NPWT system 12. As shown in FIG. 1, the dressing system 100 includes a track pad 102 and tubing that is structured to fluidly couple the dressing system 100 to a negative pressure source 14 (e.g., a vacuum pump, etc.) to communicate negative pressure to the wound site 10 through the dressing system 100. The track pad 102 is coupled to an upper cover 104 of the dressing system 100, at an intermediate position (e.g., central position, etc.) along the upper cover 104. The track pad 102 includes an airtight connection mechanism that fluidly connects a patient facing side of the upper cover 104 to the tubing.

The dressing system 100 includes a base dressing 106 (e.g., base layer, etc.) and an absorbent dressing 108 (e.g., absorbent layer, removable layer, canister, absorbent pad, etc.) detachably coupled to the base dressing 106. The absorbent dressing 108 disposed between the negative pressure source 14 and the base dressing 106 and captures any fluid (e.g., wound exudate, antiseptic, instillation fluid, etc.) that is pulled away from the wound site 10 through the base dressing 106. In other embodiments, the dressing system 100 may be used independently from the NPWT system 12, as a standalone absorbent wound dressing.

As shown in FIG. 1, the dressing system 100 also includes a touch fastener 110 that is “sandwiched” or otherwise disposed between the base dressing 106 and the absorbent dressing 108. The touch fastener 110 includes a first fastening layer 112 and a second fastening layer 114. In some embodiments, the touch fastener 110 also includes an adhesive layer (e.g., a pressure sensitive adhesive layer, etc.) to bond one or more portions of the touch fastener 110 to the base dressing 106 or absorbent dressing. In the embodiment of FIG. 1, the first fastening layer 112 is laminated to the base dressing 106. In other embodiments, the dressing system 100 may include additional, fewer, and/or different layers.

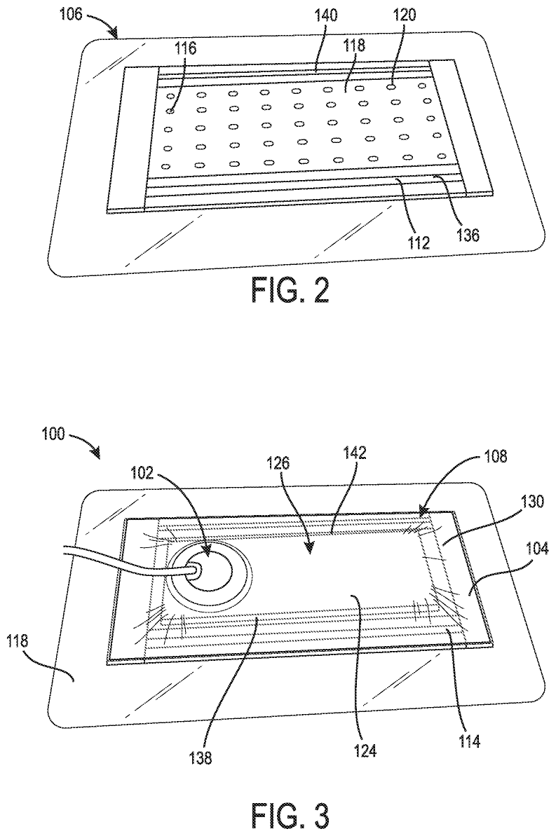

FIG. 2 shows a perspective view of the base dressing 106. The base dressing 106 is structured as a wound dressing for an open wound. The base dressing 106 includes a foam material 116 (e.g., foam manifold, foam layer, etc.) that is configured to engage (e.g., contact) the wound and to manifold fluid away from the wound. The base dressing 106 also includes a perforated layer 118 (e.g., perforated film, etc.) including a plurality of openings 120 that are configured to fluidly couple the foam material 116 with the absorbent dressing 108 (see FIG. 1). In some embodiments (e.g., as shown in FIG. 1), the perforated layer 118 is a double sided adhesive film that is separate from the base dressing 106. In other embodiments, the perforated layer 118 forms part of the base dressing 106. As shown in FIG. 2, the foam material 116 is coupled to a patient facing side of the perforated layer 118. The foam material 116 is aligned with the perforated layer 118 such that the foam material 116 is positioned below the plurality of openings 120 and substantially covers the plurality of openings 120. As shown in FIG. 2, the perforated layer 118 includes a perimeter portion that extends radially beyond the foam material 116 and that couples the base dressing 106 to the patient's tissue. In other embodiments, the base dressing 106 may be structured as an incisional wound dressing that includes a patient interface layer disposed between at least one of (i) the perforated layer 118 and the patient's tissue, and/or (ii) the foam material 116 and the patient's tissue. The perforated layer 118 may be a double-sided film that includes a pressure sensitive adhesive on both sides of the film.

As shown in FIG. 1, an inner surface 122 of the perforated layer 118 is bonded (e.g., adhered) or otherwise coupled to the first fastening layer 112. For example, the first fastening layer 112 may be laminated to the perforated layer 118. In this way, the perforated layer 118 may be permanently affixed to the first fastening layer 112 such that the first fastening layer 112 cannot be removed without damaging the perforated layer 118. In other embodiments, the first fastening layer 112 may be bonded to the perforated layer 118 using glue, epoxy, and/or another suitable adhesive product (e.g., a pressure sensitive adhesive, etc.).

As shown in FIG. 1, the second fastening layer 114 is bonded or otherwise coupled to a first side 117 (e.g., outer side, outer surface, wound-facing surface, etc.) of the absorbent dressing 108 and is permanently affixed to the absorbent dressing 108. Referring to FIG. 3, a perspective view of the dressing system 100 of FIG. 1 is shown. As shown in FIGS. 1 and 3, the absorbent dressing 108 includes an upper cover 104 and an absorbent material 124 coupled to the upper cover 104 at a central position along the upper cover 104. As shown in FIG. 3, the upper cover 104 is a film that includes a pressure sensitive adhesive on a lower portion of the film. The second fastening layer 114 may be bonded directly to the upper cover 104, along a perimeter portion of the upper cover 104, to at least partially surround the absorbent material 124. In some embodiments, the first fastening layer 112 and the second fastening layer 114 circumscribe the absorbent material 124 and the openings in the perforated layer 118. In other embodiments, the first fastening layer 112 and the second fastening layer 114 are disposed along only a perimeter portion of the absorbent material 124 and/or plurality of openings 120 (e.g., along only two sides of the dressing system 100, three sides, etc.).

As shown in FIG. 3, the upper cover 104 defines a recessed area 126 that is sized to receive the absorbent material 124 therein. In other embodiments, a separate adhesive product may be applied to the patient facing side of the upper cover 104 to permanently affix the absorbent material 124 to the upper cover 104. In yet other embodiments, the absorbent material 124 may be printed or otherwise formed onto the upper cover 104.

### Base Dressing

The base dressing 106 is configured to engage with a patient's skin or tissue in the area surrounding the wound and to form a substantially airtight seal between the wound site 10 and the external environment along a peripheral portion of the base dressing 106. As shown in FIG. 1, the outer surface 128 (e.g., patient facing surface, etc.) of the perforated layer 118 is bonded to and sealingly engaged with the wound site 10. The inner surface 122 of the perforated layer 118 is coupled to the first side 117 of the upper cover 104. The perforated layer 118 may be made from a polymer film (e.g., a polyurethane film), a medical textile (e.g., Asahi nylon), and/or another suitable material. The perforated layer 118 may include a suitable low tack adhesive (e.g., silicone or polyurethane gel) to facilitate bonding with the tissue at the wound site 10 (or surrounding the wound site 10). The adhesive may be applied to the outer surface 128 of the perforated layer 118 proximate to an outer perimeter of the perforated layer 118, and/or at any other suitable location along the outer surface 128. In some embodiments, the adhesive may be distributed evenly across the outer surface 128. In other embodiments, the perforated layer is a double-sided adhesive layer that includes a pressure sensitive adhesive on both the inner surface 122 and the outer surface 128.

In some embodiments, the perforated layer 118 may be substantially the same shape and have the same area as the upper cover 104. In other embodiments, the perforated layer 118 is a patient interface layer that extends peripherally beyond the upper cover 104. As shown in FIG. 1, the perforated layer 118 defines plurality of openings 120 that are disposed centrally along the perforated layer 118 (e.g., a central position, in the middle of the perforated layer 118, etc.). The openings 120 provide fluid communication between the wound site 10 (e.g., wound) and an absorbent material in the absorbent dressing 108. In the embodiment of FIG. 2, the plurality of openings 120 include circular openings distributed in a uniform array across a central portion of the perforated layer 118. In other embodiments, the openings 120 may be elongated slits. It will be appreciated that the size, shape, and number of openings may be different in various alternative embodiments.

The base dressing 106 also includes a foam material 116 coupled to the outer surface 128 of the perforated layer 118. The foam material 116 is structured to manifold the flow of fluid (e.g., wound exudate, distillation fluid, etc.) toward the absorbent dressing 108 (e.g., through the plurality of openings 120). The foam material 116 is aligned with the perforated layer 118 such that the foam material 116 is positioned below the plurality of openings 120 and covers at least some of the plurality of openings 120. The foam material 116 may include, but is not limited to, a cellular foam, an open-cell foam, a reticulated foam, porous tissue collections, and/or other porous materials (e.g., gauze). In some embodiments, the foam material may include an open-cell, reticulated polyurethane foam such as a GRANUFOAM™ dressing available from Kinetic Concepts. Inc. of San Antonio, Texas. In some embodiments, the retainer layer may include an open-cell, reticulated polyurethane foam such as a V.A.C. VERAFLO™ dressing available from Kinetic Concepts. Inc. of San Antonio. Texas or a V.A.C. VERAFLO CLEANSE CHOICE™ dressing (Acelity, San Antonio TX).

### Absorbent Dressing

The absorbent dressing 108 is detachably coupled to the base dressing 106 and includes the upper cover 104, the absorbent material 124, and the track pad 102 (e.g., a negative pressure interface). As shown in FIGS. 1 and 3, the upper cover 104 (e.g., top cover, etc.) substantially encloses the area above the wound site 10 and provides a fluid connection between the NPWT system (e.g., vacuum pump) and the wound site 10. In some embodiments, the upper cover 104 includes a drape 130 (see FIG. 3) and/or film layer that extends over the absorbent material 124. The upper cover 104 (e.g., drape 130) defines a recessed area 126 (e.g., pocket, cavity, etc.) at a central position along the upper cover 104 that is sized to receive the absorbent material 124 therein. In some embodiments, the recessed area 126 is a domed area formed by stretching the upper cover 104 over the absorbent material 124. The track pad 102 is coupled to the upper cover 104 at the recessed area 126 and provides the fluid connection between the upper cover 104 and the tubing for the NPWT system. The upper cover 104 may be or include a Tagaderm™ layer (e.g., a transparent or partially transparent medical dressing) made from a thin high moisture vapor transmission rate (MVTR) adhesive coated polyurethane film such as Inspire 2327/2317 or another polyurethane or polyethylene film. The upper cover 104 may be at least partially coated in adhesive, for example, on the wound-facing side of the upper cover 104 (e.g., the first side 117 of the upper cover 104), in order to bond the upper cover 104 to the second fastening layer 114 and/or the absorbent material 124.

As shown in FIG. 1, the absorbent material 124 (e.g., layer, sheet, etc.) is disposed within the recessed area 126 and coupled to the wound facing side of the upper cover 104. The absorbent material 124 is configured to absorb wound exudate and/or other fluids (e.g., instillation fluids, antiseptics, etc.) and remove them from the wound site 10. In at least one embodiment, the absorbent material 124 is made from a superabsorbent laminate (e.g., a super absorbent material) such as may be commercially available from Gelok. In other embodiments, the absorbent material 124 includes a printed absorbent polymer.

In some embodiments, the absorbent material 124 may be formed from or otherwise include a superabsorbent polymer in the form of granules. The superabsorbent polymer may include Luquasorb 1160 or 1161, such as may be commercially available from BASF. The granules may be contained in a water-soluble carrier polymer. One example of the water-soluble carrier polymer is polyvinylpyrrolidone (PVP). The superabsorbent polymer and the water-soluble polymer may be formed into a slurry or a suspension using an organic solvent. The organic solvent may include propanone or propanol and may aid in delivery of the absorbent material 124 to the upper cover 104 or another carrier (e.g., an absorbent foam manifold disposed within the recessed area 126). In some embodiments, to increase the softness of the superabsorbent granules, a plasticizer may be added to the slurry. In one embodiment, the plasticizer may be water. In some embodiments, the slurry to form the absorbent material 124 may have a formulation of 20 parts by mass of PVP, 10 parts by mass of a superabsorbent polymer, 1 part by mass of glycerol, and 100 parts by mass of propanone. In some embodiments, to plasticize the granules, 1 part to 2 parts by mass of water may be added to the slurry mixture. In other embodiments, a water-soluble polymer superabsorbent precursor, such as acrylic acid or 2-acrylamido-2-methyl-propanesulfonic acid (AMPS), with suitable UV curing additives, may replace the superabsorbent polymer. Such a precursor may be a relatively low viscosity solution and can be printed onto at least one of the upper cover 104 or a separate carrier and exposed to UV light to form a soft gel, eliminating the need for a plasticizer. In some embodiments, the water-soluble polymer superabsorbent precursor may be similar to that used for preparing hydrogel coatings.

By way of example, the slurry mixture may be applied to the wound-facing side of the upper cover 104 to form an absorbent layer. In some embodiments, the slurry may be applied to the upper cover 104 through standard printing methods, such as silk screen printing, gravure printing, or by x-y plotter printing. The absorbent layer may be applied in a variety of different shapes such as circles, squares, hexagons, hoops/halos, stars, crosses, a range of lines, or any combination of shapes. The absorbent layer may be substantially evenly distributed on the upper cover 104 within the recessed area 126. In some embodiments, the absorbent material 124 may include a flexible plasticized hydrophilic polymer matrix having a substantially continuous internal structure. In some embodiments, the absorbent material 124 may include a combination of different materials.

A thickness of the absorbent material 124 may be sized to accommodate different amounts (e.g., rates, etc.) of fluid flow from the wound site 10, without changing the position of the fastening layer on the upper cover 104. For example, a thickness of the absorbent material 124 for low exudating wounds/incisions would be sized for a maximum capacity of 50 cubic centimeters of wound exudate, although larger sizes (e.g., 100 cc. 150 cc. 200 cc, or greater) could be offered for other wound types and conditions. Because the absorbent dressing 108 is replaceable, the amount of absorbent material 124 could be varied during treatment, without having to remove the base dressing 106 and without having to change the design of the negative pressure source (e.g., vacuum pump).

As shown in FIGS. 1 and 3, the absorbent dressing 108 includes a negative pressure interface, shown as track pad 102 that fluidly couples the dressing system 100 to a NPWT system (e.g., a low pressure source such as a vacuum pump, etc.). The track pad 102 is coupled to the upper cover 104 above the absorbent material 124. As shown in FIG. 1, the track pad 102 includes a film layer 132, which may include a double-sided film with a pressure sensitive adhesive applied to both sides of the film. The film layer 132 couples the track pad to the upper cover 104, to an opening on the upper cover 104. The track pad 102 also includes a hydrophobic filter 134, which is configured to substantially prevent the flow of fluid through the track pad 102 (e.g., to block wound exudate, distillation fluids, and/or other fluids from leaving the absorbent material 124). In this way, the fluids remained trapped within the absorbent material 124 during treatment. The absorbent dressing 108 therefore functions as a dressing-mounted canister that traps the fluid from the wound. As shown in FIG. 1, the absorbent material 124 and any fluid contained within the absorbent material 124 is separated from the wound site 10 by the base dressing 106 (e.g., foam material and perforated layer). This separation reduces moisture levels at the wound site and the risk of maceration.

### Touch Fastener

The touch fastener 110 is disposed between the perforated layer 118 and the upper cover 104 and is configured to detachably couple the perforated layer 118 to the upper cover 104. The touch fastener 110 is structured to form a substantially airtight and liquid tight seal between the perforated layer 118 and the upper cover 104. In the embodiment of FIG. 1, the touch fastener 110 includes a first fastening layer 112 and a second fastening layer 114 that are structured to releasably and resealably couple the upper cover 104 (e.g., drape 130) to the perforated layer 118.

As shown in FIG. 1, the first fastening layer 112 and the second fastening layer 114 each include a plurality of lineal fastening strips that extend along the sides of the dressing system 100 and circumscribe the plurality of openings 120 in the perforated layer 118. The fastening strips of the first fastening layer 112 are substantially aligned with the fastening strips of the second fastening layer 114. In at least one embodiment, the first fastening layer 112 and the second fastening layer 114 each includes four fastening strips to encompass all four sides of the openings 120 and the absorbent material 124. In other embodiments, the shape, position, and/or number of fastening strips may be different.

In at least one embodiment, as shown in FIGS. 2-3, first fastening layer 112 and the second fastening layer 114 each include a pair of fastening strips that extend along a longest dimension of the absorbent material 124. For example, as shown in FIG. 2, the first fastening layer 112 may include only two fastening strips, including a first fastening strip 136 extending along a first side 138 of the absorbent material 124 (see FIG. 3), and a second fastening strip 140 positioned on a second side 142 (see FIG. 3) of the absorbent material 124 that is opposite from the first side 138. The first fastening strip 136 and the second fastening strip 140 may be substantially parallel to one another and may be oriented in a direction that is substantially parallel to a longest dimension of the absorbent material 124 and/or a longest dimension of the perforated layer 118. In this implementation, a thick gel adhesive may be applied to the perforated layer 118 and/or upper cover 104, along gaps formed between the ends of the first fastening strip 136 and the second fastening strip 140. The gel adhesive or other bonding agent is configured to be repositionable and replaceable. In other words, the gel adhesive may be tacky and may be pushed to other locations along the perforated layer 118 and/or pulled away from the perforated layer 118 without damaging the perforated layer 118. According to an illustrative embodiment, the gel adhesive is a hydrogel having a water activity within a range between approximately 0.45 and 0.69, a solution uptake within a range between approximately 1000% and 3000%, a tack force within a range between approximately 135 g and 194 g, and a coat weight within a range between approximately. 0.92 kg/m2 and 1.18 kg/m2. In other embodiments, the properties of the hydrogel may be different. In another embodiment, the gel adhesive may include a silicone adhesive, DermaTac™ (e.g., a silicone and/or acrylic hybrid drape), and/or a hydrophilic gel adhesive, or another suitably tacky adhesive. In yet another embodiment, the gel adhesive includes a high-tack silicone similar to or the same as 3M 2480 Hi Tack Silicon. Among other benefits, using a gel adhesive or other bonding agent in combination with the fastening strips (e.g., on a short end of the dressing system 100) reduces the force required to separate (e.g., peel back, remove, etc.) the upper cover 104 from the perforated layer 118. The arrangement of fastening strips and gel adhesive may differ in various alternative embodiments. For example, the fastening strips may be used on three sides of the perforated layer 118 and the gel adhesive may be using on the fourth, open side.

The touch fastener 110 may be any type of selectively resealable and reclosable mechanical interlock that can provide a substantially airtight and liquid tight seal between the absorbent dressing 108 (e.g., upper cover 104) and the base dressing 106 (e.g., perforated layer 118). For example, the touch fastener 110 may be a Velcro-style hook and loop fastener that includes (i) a male fastening strip and (ii) a female fastening strip that is structured to couple to the male fastening strip in response to an applied pressure between the male fastening strip and the female fastening strip. For example, in the embodiment of FIG. 1, the second fastening layer 114 may include at least one male fastening strip that is engageable with a female fastening strip of the first fastening layer 112 by pressing the male fastening strip into the female fastening strip, such that at least a portion of the male fastening strip extends into (e.g., protrudes into, is received within, etc.) the female fastening strip.

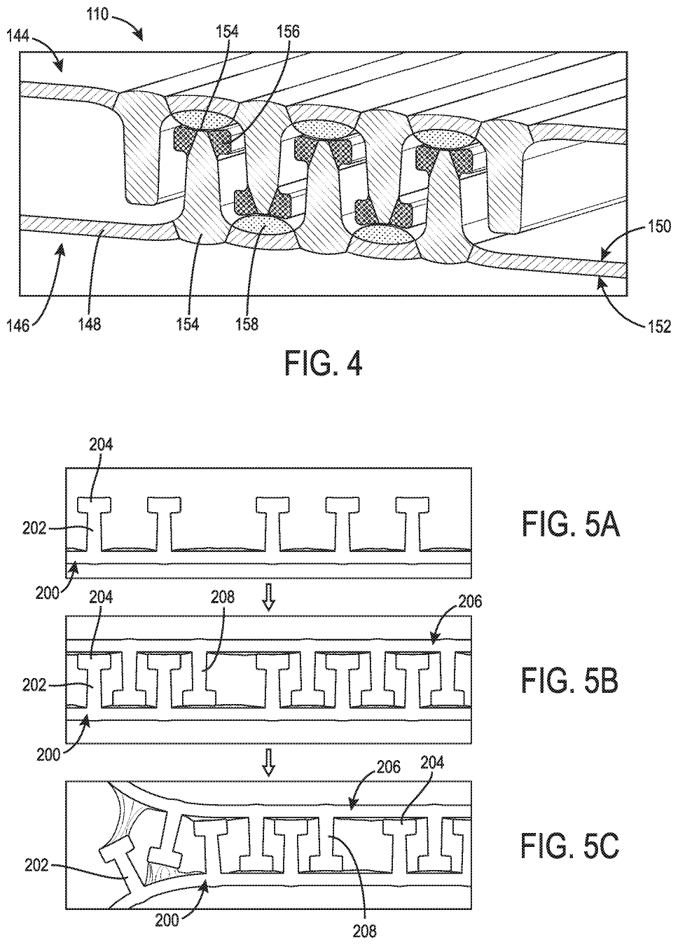

FIG. 4 shows a close up view of the joint that is formed between an upper fastening strip 144 of the first fastening layer 112 and a lower fastening strip 146 of the second fastening layer 114, according to an illustrative embodiment. As shown, upper fastening strip 144 and the lower fastening strip 146 are the same and/or have a substantially similar structure. In other embodiments, the structure of the upper fastening strip 144 and/or the lower fastening strip 146 may be different. In the embodiment of FIG. 4, the lower fastening strip 146 includes a base wall 148 having a first, outer surface 150, and a second, inner surface 152. The lower fastening strip 146 also includes a plurality of stems 154 (e.g., rails, protrusions, etc.) coupled to the base wall 148 along the first surface 150 and extending outwardly from (e.g., away from) the first surface 150 toward the upper fastening strip 144. Each stem 154 extends laterally across the first surface 150, between opposing sides of the lower fastening strip 146. In the embodiment of FIG. 4, the stems 154 are spaced in approximately equal intervals along a longitudinal direction (e.g., perpendicular to the lateral direction) between opposing ends of the lower fastening strip 146. In other embodiments, the number and arrangement of stems 154 may be different.

In some embodiments, the lower fastening strip 146 and/or upper fastening strip 144 include a hook portion (e.g., flange, ledge, etc.) coupled to an outer, free end of each stem 154. For example, as shown in FIG. 3, each stem 154 includes shoulder portions 156 (e.g., shoulders, etc.) extending outwardly from an upper end of the stem 154 at an oblique angle relative to a central axis of the stem 154. The shoulder portions 156 extend away from an upper end of the stem 154 in an at least partially longitudinal direction toward the space between adjacent stems 154. In other embodiments, the shape and/or position of the hook portion of the stem 154 may be different. For example, FIGS. 5A-5C show an example lower fastening strip 200 that includes stems 202 having an outer hook portion 204 that extends in a substantially perpendicular direction relative to the body of each stem 202 (e.g., substantially parallel to the first surface 150). Together, the stem 202 and the hook portion 204 form a substantially “T” shaped protrusion that is structured to engage and interlock with the “T” shaped protrusions of an upper fastening strip 206. In other words, the combination of the stems 202 and hook portion 204 provide a direct mechanical connection between the lower fastening strip 200 and the upper fastening strip 206. In other embodiments, the shape of the hook portion may be different. As shown in FIG. 5B, pressing the upper fastening strip 206 into the lower fastening strip 200 (e.g., against the lower fastening strip 200) engages the stems 202 with the spaces formed between adjacent ones of the stems 208 in the upper fastening strip 206. The hooked portions of the lower fastening strip 200 interlock the hooked portions of the upper fastening strip 206. According to an illustrative embodiment, the stems 202 are formed onto the fastening strips using a micro-profile extrusion process from a flexible plastic material such as polyethylene or another suitable plastic.

In at least one embodiment, the first fastening layer 112 and/or the second fastening layer 114 is a hybrid closure that includes both a non-adhesive-based mechanical fastener (e.g., stems 154) and an adhesive material interposed between adjacent mechanical fastener portions. As shown in FIG. 4, the lower fastening strip 146 and the upper fastening strip 144 each include an adhesive 158 or other bonding agent disposed on the base wall 148, within valleys between adjacent ones of the stems 154. The adhesive 158 is positioned to engage with an outer end surface 160 of a respective one of the stems from an opposing fastening strip, which helps to distribute the adhesive 158 and provides a more robust seal between the upper fastening strip 144 and the lower fastening strip 146. In other embodiments, the adhesive 158 may be applied to different areas of the fastening strips. The adhesive 158 may be a pressure sensitive adhesive material and/or another suitable adhesive product (e.g., silicone, polyurethane gel, etc.).

Among other benefits, the touch fastener 110 structure provides a resealable and reclosable connection between the base dressing 106 (e.g., perforated layer 118) and the absorbent dressing 108 (e.g., upper cover 104) (see FIG. 1). As shown in FIG. 5C, the touch fastener 207 is structured such that the upper fastening strip 206 can be pulled apart from (e.g., uncoupled from) the lower fastening strip 200 by peeling back one end of the upper fastening strip 206. This causes the stems 202 to pull away from the adhesive material and past the mechanical interlock formed by the hooked portion of the stems 202.

### Dressing Operation

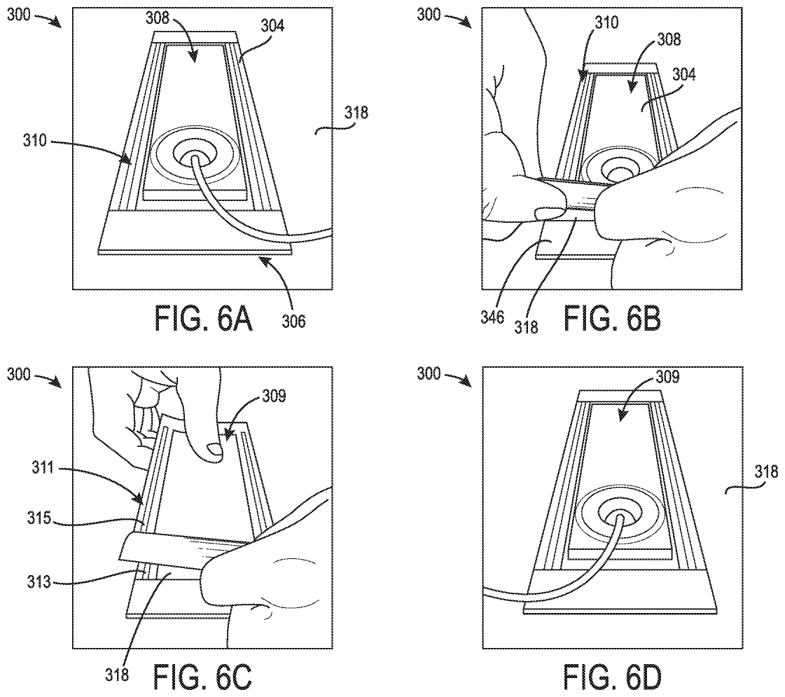

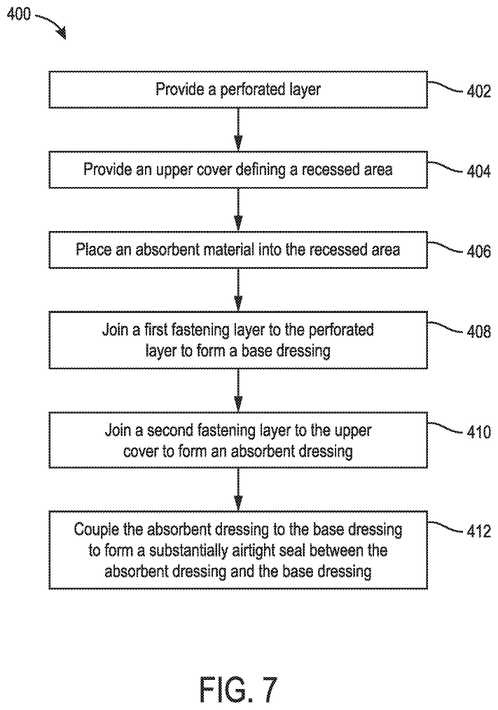

FIGS. 6A-6D show another example embodiment of a replaceable absorbent dressing system, shown as dressing system 300 in various states of use. As shown in FIG. 6A, the dressing system 300 may be provided to a clinician and/or other user as a single unitary structure, with the touch fastener 310 pre-applied in between a first absorbent dressing 308 (e.g., upper cover 304) and the base dressing 306 (e.g., perforated layer 318). Among other benefits, preassembly of the dressing system 300 ensures proper alignment between the upper fastening strips and the lower fastening strips, and the most robust connection between adjacent dressing layers.

To apply the dressing system 300 to a patient, a clinician or other user simply removes a backing layer from an outer surface of the perforated layer 318 and applies the exposed surface of the perforated layer 318 to the tissue. The negative pressure source may then be activated to reduce the pressure in the space above the wound site, in between the wound and the upper cover 304, which compresses the absorbent material. As shown in FIG. 6B, when replacement of a first (e.g., saturated) absorbent dressing 308 is required (e.g., once the absorbent material has become fully saturated with wound exudate, etc.), the clinician can stop the NPWT therapy (e.g., can deactivate the negative pressure source, decouple the pressure source from the dressing system 300, etc.), and begin peeling back the first absorbent dressing 308 (e.g., upper cover 304) from one end of the dressing system 300 (e.g., the short end as shown in FIG. 6B). In the embodiment shown, the short ends of the first absorbent dressing 308 are secured to the perforated layer 318 using a thick gel adhesive product 346. The long ends of the first absorbent dressing 308 are secured to the perforated layer 318 using the touch fastener 310. In other embodiments, the touch fastener 310 may be used without the gel adhesive, by positioning additional sections of the touch fastener 310 to the short ends of the first absorbent dressing 308 and perforated layer 318.

As shown in FIG. 6C, the clinician can select a second (e.g., new, unsaturated, etc.) absorbent dressing 309 (e.g., a second absorbent dressing, a clean absorbent dressing, etc.) having a touch fastener 311 that is positioned in the same area of second absorbent dressing 309 as the first absorbent dressing 308. This operation may include selecting a size (e.g., a thickness) of the absorbent material contained within the second absorbent dressing 309, based on an amount and/or flow rate of the wound exudate.

As shown in FIG. 6C, after obtaining a second absorbent dressing 309, the clinician can attach the second absorbent dressing 309 to the perforated layer 318 by aligning the fastening strips of both the first fastening layer 313 and the second fastening layer 315. The clinician may then press down on the second absorbent dressing 309, above each of the touch fastener strips (e.g., along a perimeter portion of the second absorbent dressing 309) to engage the fastening strips of each fastening layer and to thereby engage the mechanical connection of the fastening strips. Finally, the clinician may secure the short ends of the second absorbent dressing 309 by pressing the short ends of the second absorbent dressing 309 into the thick gel adhesive on the perforated layer 318, before restarting the NPWT therapy (e.g., activating the negative pressure source), as shown in FIG. 6D.

In some embodiments, the dressing system 300 may be used as an in-process sampling tool that facilitates determination of the health of the wound. For example, a first absorbent dressing may include a relatively small amount of absorbent material 324 (e.g., 10 cc. 20 cc, etc.), which will saturate quickly when applied to the patient. Upon removal, the wound exudate captures by the first absorbent dressing can be sampled to check for infection in the patient, to evaluate the health of the wound, and/or overall treatment progress. In this way, the clinician can take action (e.g., adjust treatment procedures, introduce distillation fluid, etc.) without having to remove and replace the entire dressing system (e.g., the base dressing). A larger second absorbent dressing (e.g., 50 cc or greater) can be installed in place of the first absorbent dressing to increase the time between replacement after analyzing the fluids from the wound.

### Method of Making a Replaceable Absorbent Dressing System

Referring to FIG. 9, a flow diagram of a method 400 of making a replaceable absorbent dressing is shown, according to an illustrative embodiment. The method 400 includes providing a perforated layer, at operation 402. Operation 402 may include providing a double-sided film layer such as a thin film layer, at operation 402. Operation 402 may further include cutting a plurality of openings (e.g., holes) in the perforated layer, along a central portion of the perforated layer. Operation 402 may further include applying an adhesive product (e.g., a pressure sensitive adhesive) to at least one side of the perforated layer. In some embodiments, and particularly for incisional wounds, the method may further include applying a second film layer (e.g., a double sided film with pressure sensitive adhesive) to an underside of the perforated layer to serve as a patient interface layer, and/or forming an opening in the second film layer, at a central location along the second film laver.

Operation 402 may also include providing a foam material and joining the foam material to a patient facing side of the perforated layer to form a base dressing. Operation 402 may include aligning the foam material with the perforated layer such that the foam material is positioned below at least some of the plurality of openings in the perforated layer.

The method 400 also includes providing an upper cover defining a recessed area, at operation 404. Operation 404 may include cutting a drape made from Tagaderm™ or another thin film layer to substantially match the size of the perforated layer, or such that an outer perimeter edge of the film layer protrudes just beyond the foam material in the base dressing. Operation 404 may further include forming a recessed area into the drape using a press operation and/or a heat treating operation and/or by stretching the drape over an absorbent material. Operation 404 may also include incorporating a negative pressure coupling (e.g., track pad, etc.) into the drape. For example, operation 404 may include joining a track pad to the drape using a double-sided adhesive film layer. Operation 404 may also include joining a hydrophobic filter to the track pad.

In operation 406, an absorbent material is placed into the recessed area of the upper cover. Operation 406 may include providing the absorbent material and aligning the absorbent material with the recessed area such that the absorbent material is positioned above at least some of the plurality of openings in the perforated layer. Operation 406 may include joining the absorbent material to the upper cover, for example, using an adhesive product or via the pressure sensitive adhesive on the upper cover.

In operation 408, a first fastening layer is joined to the perforated layer (e.g., the patient interface layer, etc.). Operation 408 may include providing the first fastening layer; for example, by providing a plurality of fastening strips, and applying an adhesive product to a backing of the fastening strips and/or to select areas of the perforated layer (e.g., along a perimeter region of the plurality of openings). In other embodiments, operation 408 may include laminating the first fastening layer to the perforated layer. Operation 408 may further include applying the fastening strips of the first fastening layer to the perforated layer. In at least one embodiment, operation 408 includes applying fastening strips along the perimeter region of the perforated layer just outside of the plurality of openings.

In other embodiments, operation 408 includes applying the fastening strips along only a perimeter portion of the perforated layer. For example, operation 408 may include positioning a first fastening strip of the first fastening layer along a direction that is substantially parallel to a longest dimension of the perforated layer and adhering the first fastening strip to the perforated layer on a first side of the plurality of openings. Operation 408 may further include adhering a second fastening strip to a second side of the perforated layer opposite from the first side. Operation 408 may additionally include applying a gel adhesive to the perforated layer to substantially fill a gap between the first fastening strip and the second fastening strip (e.g., between ends of the first fastening strip and the second fastening strip, such that the fastening strips and gel adhesive together circumscribe the plurality of openings).

In operation 410, a second fastening layer is joined to the upper cover to form an absorbent dressing. Operation 410 may include providing the second fastening layer: for example, by providing a plurality of fastening strips, and applying an adhesive product to a backing of the fastening strips and/or to select areas of the drape that are aligned with the first fastening layer. Operation 410 may further include applying (e.g., adhering) the fastening strips of the second fastening layer to the drape.

In operation 412, the absorbent dressing is coupled to the base dressing to form a substantially airtight seal between the absorbent dressing and the base dressing. Operation 412 may include aligning the first fastening layer with the second fastening layer and pressing (e.g., via a press or another suitable fixture or process) a male interlocking member of the second fastening layer into a female interlocking member of the first fastening layer (e.g., by engaging the stems, rails, or other mechanical interlock between adjacent fastening strips).

In at least one embodiment, the second fastening layer is coupled to the first fastening layer before joining the second fastening layer to the upper cover. Beneficially, this modified order of operations ensures alignment is maintained between the first and second fastening layers during manufacturing. In other embodiments, the method 400 may include additional, fewer, and/or different operations.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

What is claimed is:

1. A replaceable absorbent dressing system, comprising:
a dressing including a perforated layer; and
an absorbent pad detachably coupled to the dressing such that the absorbent pad can be separated from and reattached to the dressing while still forming a substantially airtight seal with the dressing.

2. The replaceable absorbent dressing system of claim 1, wherein the dressing further includes a foam material disposed on a skin facing side of the perforated layer.

3. The replaceable absorbent dressing system of claim 1, wherein the absorbent pad is detachably coupled to the dressing by intervening fastening layers including a first fastening layer and a second fastening layer that is detachably coupled to the first fastening layer.

4. The replaceable absorbent dressing system of claim 3, wherein the first fastening layer includes a strip extending along a perimeter portion of the absorbent pad, the strip comprising a plurality of stems that are configured to mechanically fasten the first fastening layer to the second fastening layer when the first fastening layer is pressed against the second fastening layer.

5. The replaceable absorbent dressing system of claim 3, wherein the first fastening layer includes a female interlocking member, and wherein the second fastening layer includes a male interlocking member configured to sealingly engage with the female interlocking member.

6. The replaceable absorbent dressing system of claim 3, wherein the first fastening layer comprises a first fastening strip and a second fastening strip positioned on an opposite side of the absorbent pad as the first fastening strip, and wherein the first fastening strip and the second fastening strip are each oriented in a direction that is substantially parallel to a longest dimension of the absorbent pad.

7. The replaceable absorbent dressing system of claim 1, wherein the absorbent pad forms part of an absorbent dressing, the absorbent dressing further comprising:
an upper cover coupled to the absorbent pad and covering an upper surface of the absorbent pad; and
a track pad coupled to the upper cover and fluidly coupled to an area of the upper cover that contains the absorbent pad.

8. The replaceable absorbent dressing system of claim 7, wherein the track pad further includes a hydrophobic filter that substantially prevents fluid from passing through the track pad.

9. The replaceable absorbent dressing system of claim 1, wherein the perforated layer comprises a plurality of openings that fluidly couple the absorbent pad with a patient facing side of the dressing.

10. The replaceable absorbent dressing system of claim 9, wherein the absorbent pad is aligned with the perforated layer such that the absorbent pad is positioned above at least some of the plurality of openings.

11. The replaceable absorbent dressing system of claim 1, wherein the absorbent pad is detachably coupled to the dressing by a gel adhesive.

12. A negative pressure wound therapy system, comprising:
a first dressing including an absorbent material and a negative pressure interface;
a second dressing including a perforated layer;
a touch fastener disposed between the first dressing and the second dressing, the touch fastener configured to detachably couple the first dressing to the second dressing and form a substantially airtight seal between the first dressing and the second dressing; and
a negative pressure source coupled to the negative pressure interface.

13. The negative pressure wound therapy system of claim 12, wherein the second dressing further includes a foam material disposed on a skin facing side of the perforated layer.

14. The negative pressure wound therapy system of claim 12, wherein the absorbent material forms part of an absorbent dressing, the absorbent dressing further comprising:
an upper cover coupled to the absorbent material and covering an upper surface of the absorbent material; and
a track pad coupled to the upper cover and fluidly coupled to an area of the upper cover that contains the absorbent material.

15. The negative pressure wound therapy system of claim 14, wherein the track pad further includes a hydrophobic filter that substantially prevents fluid from passing through the track pad.

16. The negative pressure wound therapy system of claim 12, wherein the perforated layer comprises a plurality of openings that fluidly couple the absorbent material with a patient facing side of the second dressing.

17. A replaceable absorbent dressing, comprising:
an upper cover defining a recessed area on a patient facing side of the upper cover;
an absorbent material disposed within the recessed area; and
a touch fastener coupled to the upper cover along a perimeter portion of the upper cover, the touch fastener configured to detachably couple the upper cover to a dressing and form a substantially airtight seal between the upper cover and the dressing.

18. The replaceable absorbent dressing of claim 17, wherein the upper cover further includes a negative pressure interface, and wherein the replaceable absorbent dressing further includes a track pad coupled to the negative pressure interface.

19. The replaceable absorbent dressing of claim 18, wherein the track pad includes a hydrophobic filter that substantially prevents fluid from passing through the track pad.

20. The replaceable absorbent dressing of claim 17, wherein the touch fastener comprises a first fastening strip and a second fastening strip positioned on an opposite side of the absorbent material as the first fastening strip, and wherein the first fastening strip and the second fastening strip are each oriented in a direction that is substantially parallel to a longest dimension of the absorbent material.
